(19) United States
(12) Patent Application Publication
(10) Pub. No.: US 2009/0030077 A1
(43) Pub. Date: Jan. 29, 2009

(54) Novel Fenofibrate Formulations and Related Methods of Treatment

(75) Inventors: Orn Almarsson, Shrewsbury, MA (US); Pasut Ratanabanangkoon, Jatujak Bangkok (TH); Julius Remenar, Framingham, MA (US); Hector Guzman, Jamaica Plain, MA (US)

Correspondence Address: RatnerPrestia-J&J, P.O. Box 980, Valley Forge, PA, 19482-0980, US

(73) Assignee: Transform Pharmaceuticals Inc., Lexington, MA (US)

(21) Appl. No.: 12/184,864

(22) Filed: Aug. 1, 2008

Related U.S. Application Data
(63) Continuation of application No. PCT/US2007/061356, filed on Jan. 31, 2007.
Application No. PCT/US2007/061356 is a continuation-in-part of application No. 11/573,237.
(60) Provisional application No. 60/764,130, filed on Feb. 1, 2006.
(60) Provisional application No. 60/687,704, filed on Jun. 6, 2005.
(60) Provisional application No. 60/655,762, filed on Feb. 24, 2005.
(60) Provisional application No. 60/604,861, filed on Aug. 27, 2004.
(60) Provisional application No. 60/599,320, filed on Aug. 6, 2004.

(51) Int. Cl. A61K 31/235; C07C 69/616; A61P 9/00; A61P 3/00

(52) U.S. Cl. 514/543; 560/52

(57) ABSTRACT

The invention provides novel omega-3 oil liquid formulations of fenofibrate. These formulations can be substantially free of any food effect, effective in small volumes, and readily bioavailable. Notably, because the formulations of the invention contain an omega-3 oil as the major ingredient, they not only provide anithypercholesterolemic and antihypertriglyceridemic effects due to the fenofibrate active ingredient, they also provide recommended daily dosages of omega-3 oils (i.e., approximately 1 gram of omega-3 oil per day), or a portion thereof.

### FIELD OF THE INVENTION

The invention provides novel omega-3 oil liquid formulations comprising fenofibrate. These formulations can be substantially free of food effect, effective in small volumes, and readily bioavailable.

The invention also provides novel fenofibrate formulations in which fenofibrate is dissolved in a vehicle comprising an omega-3 oil, an alcohol, and a surfactant.

### BACKGROUND OF THE INVENTION

Fenofibrate (2-[4-(4-chlorobenzoyl)phenoxy]-2-methyl-propanoic acid 1-methylethyl ester) is an approved substance for the treatment of hypercholesterolemia and hypertriglyceridemia. Fenofibrate is practically insoluble in water. It is normally poorly and variably absorbed in the fasted state and currently is prescribed to be taken with food.

Known fenofibrate dosage forms include Tricor® micronized tablets in which fenofibrate powder is co-micronized with a solid wetting agent such as sodium lauryl sulfate.

The hypotriglyceridemic effects of omega-3 oils from fish oils are well established. Amounts both above and below about 1 gram per day of omega-3 oils from fish oil have been shown to decrease serum triglyceride concentrations by about 25% to about 40%, decrease VLDL blood plasma levels, and to increase both LDL and HDL plasma levels (See e.g., Harris, William S, Clin. Cardiol. 22, (Suppl. II), II-40-II-43 (1999)).

A pharmaceutical formulation comprising the beneficial effects of fenofibrate and omega-3 oil could enable both ease of administration and could improve patient compliance where both fenofibrate and omega-3 oil are suitable. In addition, the omega-3 oil may lead to even further therapeutic effect than with fenofibrate alone.

### SUMMARY OF THE INVENTION

The invention provides novel omega-3 oil liquid formulations and medicaments of fenofibrate. These formulations are effective in small volumes. Notably, because the formulations and medicaments of the invention contain an omega-3 oil as the major ingredient, they not only provide antihypertriglyceridemic and antihypercholesterolemic effects due to the fenofibrate active ingredient, they also provide recommended daily dosages of omega-3 oils (i.e., one gram of omega-3 oil per day, as per AHA guidelines), or a portion thereof.

The invention also provides novel liquid fenofibrate formulations in which fenofibrate is dissolved in a vehicle comprising an omega-3 oil, a C₁ to C₄ alcohol, and, optionally, a surfactant.

Because of their homogeneity, high potency, and minimal effective volumes, formulations of the invention can be administered in a dosage form consisting of one or two capsules as defined hereinafter and at least about 400, 450, 500, 600, 700, 800, 900, or 1000 mg per capsule or per dose of an omega-3 oil.

In one embodiment, formulations of the invention comprise an omega-3 oil, wherein the omega-3 oil is an omega-3 alkyl ester, such as an omega-3 ethyl ester. In another embodiment, formulations of the invention further comprise an omega-3 mono-, di-, or triglyceride oil.

In another embodiment, the invention provides a liquid formulation comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate. In another embodiment, the formulation comprises about 75.00, 76.00, 77.00, 78.00, 79.00, or 80.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol, and about 6.00, 7.00, 8.00, 9.00, 10.00, 11.00, or 12.00% by weight of fenofibrate.

In another embodiment, the invention provides a liquid formulation comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol, about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate.

In another embodiment, a liquid formulation of the invention comprises a mixture of fenofibrate dissolved in a vehicle comprising an omega-3 ester or omega-3 alkyl ester and a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol, and (iv) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant; and
(b) the solubility of the fenofibrate in the vehicle is about 50, 60, 70, 80, 90, 100, 110, 120, 130, 140, 150, 160, 170, 180, 190, 200, 210, 220, 230, 240, 250, 260, 270, 280, 290, or 300 milligrams per milliliter at 25 degrees C.

In another embodiment, the present invention provides a novel polymorph of fenofibrate.

In another embodiment, the present invention provides a method of making a polymorph of fenofibrate, comprising:
(a) combining fenofibrate with one or more components so as to form a solution of fenofibrate;
(b) decreasing the temperature of said solution; and
(c) collecting a precipitated solid.

The invention provides novel surfactant-containing and surfactant-free, omega-3 oil liquid medicaments of fenofibrate.

In another embodiment, the present invention provides a novel polymorph of fenofibrate.

In another embodiment, the present invention provides a method of making a polymorph of fenofibrate, comprising:
(a) combining fenofibrate with one or more components so as to form a solution of fenofibrate;
(b) decreasing the temperature of said solution; and
(c) collecting a precipitated solid.

These and other embodiments are described in greater detail in the following detailed description.

### BRIEF DESCRIPTION OF THE FIGURES

FIG. 1 shows a PXRD diffractogram of a fenofibrate polymorph (Form II).

FIG. 2 shows a semi-log plot of the mean plasma concentration of fenofibric acid in humans following oral administration.

FIG. 3 shows fenofibrate solubility as a function of ethanol concentration.

FIG. 4 shows fenofibrate solubility as a function of temperature.

FIG. 5 shows the temperature dependence of fenofibrate solubility in 85:15 E681010:Ethanol.

### DETAILED DESCRIPTION OF THE INVENTION

The invention provides novel omega-3 oil formulations of fenofibrate. These formulations are effective in small volumes. Notably, because the formulations of the invention contain an omega-3 oil as the major ingredient, they not only provide an antihypercholesterolemic effect and an antihypertriglyceridemic effect due to the fenofibrate active ingredient, they also provide recommended daily dosages of omega-3 oils (i.e., one gram of omega-3 oil per day, as per AHA guidelines), or a portion thereof.

The invention also provides novel fenofibrate formulations in which fenofibrate is dissolved in a vehicle comprising an omega-3 oil, a C₁ to C₄ alcohol, and, optionally, a surfactant.

“C₁ to C₄ alcohols” include, but are not limited to, methanol, ethanol, propanol, butanol, isopropanol, isobutanol, tert-butanol, glycerol, and propylene glycol.

An “omega-3 oil” is any oil comprising omega-3 fatty acids, omega-3 mono-, di-, or triglycerides, or omega-3 esters including, but not limited to, omega-3 alkyl esters. Omega-3 oils can be characterized using two unique descriptors, species and component. The species of an omega-3 oil is determined by the structure of the polyunsaturated carbon chain bound to the carboxyl group. The component of an omega-3 oil is determined by the chemical nature of the carboxyl group. For example, omega-3 fatty acids employ a —COOH structure bound to the polyunsaturated carbon chain, omega-3 esters employ a —COOR structure bound to the polyunsaturated carbon chain, and omega-3 mono- di- or tri-glycerides employ a —COOR′ structure bound to the polyunsaturated carbon chain, where R′ comprises a glycerol backbone. Oil composition can be described as both the species and the component(s) of an oil. For example, E681010 comprises about 68% EPA and about 10% DHA (mass percent) as ethyl esters. The remaining portion consists essentially of omega-3 oils other than EPA and DHA and other non-omega-3 oils. Such omega-3 oils can be found in, for example, fish oil, marine mammal fat, cod liver oil, walnuts and walnut oil, wheat germ oil, rapeseed oil, soybean lecithin derived oils, soybean derived oils, tofu derived oils, common bean derived oils, butternut derived oils, seaweed derived oils, flax-borage oil, and flax seed oil. Several omega-3 oils which can be used in making formulations of the invention include, but are not limited to, omega-3 oils such as Omegabrite® (Omega Natural Science), Epanova™ (Tillotts Pharma AG), OMEGA-3/90 (K D Pharma), Epax® (Pronova Biocare AS), and Incromega (Croda/Bioriginal).

“EPA” is defined as eicosapentaenoic acid (C20:5), and “DHA” is defined as docosahexaenoic acid (C22:6). Both EPA and DHA denote only the species of omega-3 oil and do not describe whether the components of such oils exist as, for example, triglycerides, diglycerides, monoglycerides, free acids, esters, or salts.

Specific omega-3 alkyl esters include the ethyl esters of EPA and DHA. For example, the E681010, OMEGA-3/90 (K D Pharma), and Incromega (Croda/Bioriginal) omega-3 ethyl esters are potential omega-3 alkyl esters.

Liquid formulations and medicaments may be described as mixtures of two or more components “by volume,” which is herein defined as the volume due to one component divided by the volume of all components of the formulation. This ratio may be converted to or reported as a percentage of the total formulation volume. Such a quantity may also be indicated by “v/v” or “percent v/v.” Similarly, the phrases “by weight” and “by mass” describe the weight or mass due to one component divided by the weight or mass of all components of the formulation. This ratio may be converted to or reported as a percentage of the total formulation weight or mass. Such a quantity may also be indicated by “w/w”, “mass percent,” or “percent w/w.”

The term “E107104” is used to describe an omega-3 oil which has a composition comprising 9.7% EPA, 71.4% DHA, and about 3.9% other omega-3 oils (mass percent) where the EPA, DHA, and other omega-3 oils are ethyl esters.

The term “E970002” is used to describe an omega-3 oil which has a composition comprising 97% EPA and about 2% other omega-3 oils (mass percent) where the EPA and other omega-3 oils are ethyl esters.

The term “TG361724” is used to describe an omega-3 oil which has a composition comprising 36% EPA (expressed as mass percent of free fatty acids), 17% DHA (expressed as mass percent of free fatty acids), and about 24% other omega-3 oils (mass percent) where the EPA, DHA, and other omega-3 oils are triglycerides.

The term “E351923” is used to describe an omega-3 oil which has a composition comprising 35% EPA (expressed as mass percent of free fatty acids), 19% DHA (expressed as mass percent of free fatty acids), and about 23% other omega-3 oils (mass percent) where the EPA, DHA, and other omega-3 oils are ethyl esters.

The term “E681010” is used to describe an omega-3 oil which has a composition comprising 67.8 percent EPA (mg/g), 9.9 percent DHA (mg/g), and about 9.6 percent other omega-3 oils (mg/g), where the EPA, DHA, and other omega-3 oils are ethyl esters.

A “liquid formulation” refers to a mixture wherein the majority of the API (active pharmaceutical ingredient) is in solution at equilibrium. For example, at least about 55.00, 60.00, 65.00, 70.00, 75.00, 80.00, 85.00, 90.00, 95.00, 96.00, 97.00, 98.00, 99.00, 99.50, or 99.99 percent of the fenofibrate in the liquid formulation is present in solution at equilibrium. Liquid formulations include, but are not limited to, semi-solid formulations.

The terms “physically stable” or “physical stability” refer to a liquid formulation of an API at equilibrium in which no crystals are present.

The terms “chemically stable” or “chemical stability” refer to a liquid formulation where there is a ≦3.0 percent loss of fenofibrate potency (recovered fenofibrate content) after 2 years at 25 degrees C.

“Surfactants” refer to a surface active compound which can alter the surface tension of a liquid in which it is dissolved and includes, but is not limited to, polyoxyl 35 castor oil and sorbitan monolaurate.

Liquid formulations and methods of the present invention can also be used with fibrates other than fenofibrate, such as clofibrate, bezafibrate, ciprofibrate, beclofibrate, etofibrate, and gemfibrozil.

Liquid formulations of the present invention can, optionally, include non-omega-3 oils. For example, one or more non-omega-3 oils can be used in combination with or in place of one or more omega-3 oils in the vehicle for fenofibrate solubilization.

In some embodiments, a liquid formulation of the present invention may be substantially homogeneous. In some embodiments, a liquid formulation may be homogeneous. In some embodiments, a liquid formulation may be a homogeneous liquid solution.

In another embodiment, an omega-3 oil contains a low percentage of non-omega-3 oil. According to the present invention, an omega-3 oil has a low percentage of non-omega-3 oil when it comprises less than about 25.00, 24.00, 23.00, 22.00, 21.00, 20.00, 19.00, 18.00, 17.00, 16.00, 15.00, 14.00, 13.00, 12.00, 11.00, 10.00, 9.00, 8.00, 7.00, 6.00, 5.00, 4.00, 3.00, 2.00, or 1.00 percent w/w non-omega-3 oil. For example, an omega-3 ethyl ester can comprise about 90 percent omega-3 ethyl esters and about 10 percent non-omega-3 ethyl esters.

The purity of omega-3 oil is an important aspect of the present invention. Oil purity is defined as a percentage (e.g., by volume or by weight) of one component of the oil with respect to the entire oil composition. For example, an ester oil with a purity of 95 percent by weight comprises at least 95 percent w/w esters. The remaining percentage may comprise free acids, mono- di- and/or triglycerides, or other components. As another example, an omega-3 ester oil with a purity of 90 percent by weight comprises at least 90 percent omega-3 esters and the remaining percentage can comprise any one or more of other oil components. A mixture of species of one component (e.g., C₈ and C₁₀ esters) need not be discerned in the determination of purity. However, a distinction of specific species within a component (e.g., C₈ and C₁₀ esters) can also be included in specific embodiments of the present invention.

In another embodiment, omega-3 oils with a purity greater than about 85.00 percent, 90.00 percent, 91.00 percent, 92.00 percent, 93.00 percent, 94.00 percent, 95.00 percent, 96.00 percent, 97.00 percent, 98.00 percent, 99.00 percent or more can be used, for example, in a liquid formulation. Omega-3 oils, specifically with a high purity of omega-3 esters, can be used. According to the present invention, omega-3 oils with a high purity comprise greater than about 85.00 percent, 90.00 percent, 91.00 percent, 92.00 percent, 93.00 percent, 94.00 percent, 95.00 percent, 96.00 percent, 97.00 percent, 98.00 percent, 99.00 percent or more of one component by weight or by volume. Omega-3 esters include, but are not limited to, esters of EPA and DHA. Omega-3 esters also include omega-3 ethyl esters.

Mixtures of omega-3 alkyl esters with other components of omega-3 oil (e.g., fatty acids, triglycerides) are not preferred according to the present invention. Fenofibrate solubility is shown, herein, to be maximized in omega-3 alkyl esters. Oils containing pure and substantially pure alkyl esters are described in the present invention.

In another embodiment, the purity of omega-3 esters or omega-3 alkyl esters is at least about 50.00 percent by weight, at least about 60.00 percent by weight, at least about 70.00 percent by weight, at least about 75.00 percent by weight, at least about 80.00 percent by weight, or at least about 85.00 percent by weight. In another embodiment, the purity of omega-3 esters or omega-3 alkyl esters is about 25.00, 30.00, 35.00, 40.00, 45.00, 50.00, 55.00, 60.00, 65.00, 70.00, 75.00, 80.00, 85.00, 90.00, 95.00, 99.00 percent or more by weight. In another embodiment, the purity of omega-3 esters or omega-3 alkyl esters is between about 25.00 and about 100.00 percent by weight, between about 40.00 and about 100.00 percent by weight, between about 50.00 and about 100.00 percent by weight, between about 60.00 and about 100.00 percent by weight, between about 70.00 and about 100.00 percent by weight, between about 75.00 and about 100.00 percent by weight, between about 75.00 and about 95.00 percent by weight, between about 75.00 and about 90.00 percent by weight, or between about 80.00 and about 85.00 percent by weight. In another embodiment, the purity of omega-3 esters or omega-3 alkyl esters is about 100.00 percent by weight, about 99.00 percent by weight, about 96.00 percent by weight, about 92.00 percent by weight, about 90.00 percent by weight, about 85.00 percent by weight, about 80.00 percent by weight, about 75.00 percent by weight, about 70.00 percent by weight, about 65.00 percent by weight, about 60.00 percent by weight, about 55.00 percent by weight, or about 50.00 percent by weight.

In another embodiment, an omega-3 oil composition comprising EPA and DHA is at least about 50.00 percent by weight, at least about 60.00 percent by weight, at least about 70.00 percent by weight, at least about 75.00 percent by weight, at least about 80.00 percent by weight, or at least about 84.00 percent by weight. In another embodiment, an omega-3 oil composition comprising EPA and DHA is about 25.00, 30.00, 35.00, 40.00, 45.00, 50.00, 55.00, 60.00, 65.00, 70.00, 75.00, 80.00, 85.00, 90.00, or 95.00 percent by weight. In another embodiment, an omega-3 oil composition comprising EPA and DHA is between about 25.00 and about 95.00 percent by weight, between about 40.00 and about 95.00 percent by weight, between about 50.00 and about 95.00 percent by weight, between about 60.00 and about 95.00 percent by weight, between about 70.00 and about 95.00 percent by weight, between about 75.00 and about 95.00 percent by weight, between about 75.00 and about 90.00 percent by weight, between about 75.00 and about 85.00 percent by weight, or between about 80.00 and about 85.00 percent by weight. In another embodiment, an omega-3 oil composition comprising EPA and DHA is about 99.00 percent by weight, about 96.00 percent by weight, about 92.00 percent by weight, about 90.00 percent by weight, about 84.00 percent by weight, about 80.00 percent by weight, about 75.00 percent by weight, about 70.00 percent by weight, about 65.00 percent by weight, about 60.00 percent by weight, about 55.00 percent by weight, or about 50.00 percent by weight.

In another embodiment, an omega-3 ester or omega-3 alkyl ester has about a 23:19 ratio of EPA:DHA, about a 75:11 ratio of EPA:DHA, about a 95:1 ratio of EPA:DHA, about a 9:2 ratio of EPA:DHA, about a 10:1 ratio of EPA:DHA, about a 5:1 ratio of EPA:DHA, about a 3:1 ratio of EPA:DHA, about a 2:1 ratio of EPA:DHA, about a 1:1 ratio of EPA:DHA, about a 1:2 ratio of EPA:DHA, about a 1:3 ratio of EPA:DHA, or about a 1:5 ratio of EPA:DHA. In another embodiment, an omega-3 ester or omega-3 alkyl ester has about a 95:1 ratio of EPA:DHA, about a 75:1 ratio of EPA:DHA, about a 50:1 ratio of EPA:DHA, about a 25:1 ratio of EPA:DHA, about a 20:1 ratio of EPA:DHA, about a 15:1 ratio of EPA:DHA, about a 10:1 ratio of EPA:DHA, about a 7.5:1 ratio of EPA:DHA, about a 5:1 ratio of EPA:DHA, about a 4:1 ratio of EPA:DHA, about a 3:1 ratio of EPA:DHA, about a 2:1 ratio of EPA:DHA, about a 1.5:1 ratio of EPA:DHA, about a 1:1 ratio of EPA:DHA, about a 1:1.5 ratio of EPA:DHA, about a 1:2 ratio of EPA:DHA, about a 1:3 ratio of EPA:DHA, or about a 1:5 ratio of EPA:DHA. In another embodiment, an omega-3 ester or omega-3 alkyl ester has from about a 95:1 ratio to about a 1:5 ratio of EPA:DHA, from about a 50:1 ratio to about a 1:1 ratio of EPA:DHA, from about a 25:1 ratio to about a 1:1 ratio of EPA:DHA, from about a 10:1 ratio to about a 1:1 ratio of EPA:DHA, from about a 5:1 ratio to about a 1:1 ratio of EPA:DHA, from about a 3:1 ratio to about a 1:1 ratio of EPA:DHA, from about a 2:1 ratio to about a 1:1 ratio of EPA:DHA, or from about a 1.5:1 ratio to about a 1:1 ratio of EPA:DHA. In another embodiment, an omega-3 ester or omega-3 alkyl ester has at least about a 1:5 ratio of EPA:DHA, at least about a 1:1 ratio of EPA:DHA, at least about a 1.5:1 ratio of EPA:DHA, at least about a 2:1 ratio of EPA:DHA, at least about a 3:1 ratio of EPA:DHA, at least about a 5:1 ratio of EPA:DHA, or at least about a 10:1 ratio of EPA:DHA.

An alcohol content of about 10.00, 15.00, 20.00, 25.00, 30.00, 35.00, or 40.00 percent by volume is presently shown to enhance fenofibrate solubilization. For example, such an alcohol is ethanol. Another alcohol is glycerol. Alcohols may have one, two, or three or more —OH groups per molecule.

Unless otherwise indicated, reports and discussions herein of fenofibrate solubility in solvents, mixtures, and liquid formulations of the invention are considered to be at 25 degrees C.

In another embodiment, the present invention provides a method for increasing the solubility of fenofibrate in an omega-3 oil, comprising adding an alcohol to said omega-3 oil.

In another embodiment, the fenofibrate solubility in a liquid formulation comprising an omega-3 oil and fenofibrate is increased by at least 10.00 percent by incorporating at least about 5.00, 10.00, 15.00, 20.00, 25.00, 30.00, 35.00, or 40.00 percent by volume of a C₁ to C₄ alcohol. In another embodiment, the fenofibrate solubility in a liquid formulation comprising an omega-3 oil and fenofibrate is increased by at least 20.00 percent by incorporating at least about 5.00, 10.00, 15.00, 20.00, 25.00, 30.00, 35.00, or 40.00 percent by volume of a C₁ to C₄ alcohol. In another embodiment, the fenofibrate solubility in a liquid formulation comprising an omega-3 oil and fenofibrate is increased by at least 30.00 percent by incorporating at least about 5.00, 10.00, 15.00, 20.00, 25.00, 30.00, 35.00, or 40.00 percent by volume of a C₁ to C₄ alcohol. In another embodiment, the fenofibrate solubility in a liquid formulation comprising an omega-3 oil and fenofibrate is increased by at least 40.00 percent by incorporating at least about 5.00, 10.00, 15.00, 20.00, 25.00, 30.00, 35.00, or 40.00 percent by volume of a C₁ to C₄ alcohol. In another embodiment, the fenofibrate solubility in a liquid formulation comprising an omega-3 oil and fenofibrate is increased by at least 50.00 percent by incorporating at least about 5.00, 10.00, 15.00, 20.00, 25.00, 30.00, 35.00, or 40.00 percent by volume of a C₁ to C₄ alcohol. In another embodiment, the fenofibrate solubility in a liquid formulation comprising an omega-3 oil and fenofibrate is increased by at least 60.00 percent by incorporating at least about 5.00, 10.00, 15.00, 20.00, 25.00, 30.00, 35.00, or 40.00 percent by volume of a C₁ to C₄ alcohol.

In another embodiment, a liquid formulation of the present invention comprises at least about 5 percent by weight of a C₁ to C₄ alcohol. For example, a liquid formulation of the present invention comprises at least about 5, 6, 7, 8, 9, 10, 11, 12, 13, 14, or 15 percent by weight of a C₁ to C₄ alcohol.

In another embodiment, a liquid formulation comprises an omega-3 oil, fenofibrate, and an amount of a C₁ to C₄ alcohol sufficient to increase the solubility of said fenofibrate by at least about 2.50 percent, 5.00 percent, 10.00 percent, 15.00 percent, 20.00 percent, 25.00 percent, 30.00 percent, 35.00 percent, 40.00 percent, 45.00 percent, 50.00 percent, 55.00 percent, or 60.00 percent over that of the same formulation without alcohol.

It has also been discovered that a particular form (component) of omega-3 oil is superior in solubilizing fenofibrate. Esters of omega-3 oil have shown greater solubilization power than other forms of omega-3, such as triglycerides. As shown in the exemplification, omega-3 alkyl esters have shown higher solubility of fenofibrate. The employment of both omega-3 alkyl esters and an alcohol in a liquid formulation of the present invention have shown greatly unexpected improvements in fenofibrate solubility. The total amount of EPA and DHA is a factor influencing the solubility of fenofibrate. An increase in the amount of EPA and DHA in a liquid formulation results in an increase in fenofibrate solubility.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about 100, 101, 102, 103, 104, 105, 106, 107, 108, 109, or 110 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about 100, 101, 102, 103, 104, 105, 106, 107, 108, 109, 110, 111, 112, 113, 114, 115, 116, 117, 118, 119, or 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about 110, 111, 112, 113, 114, 115, 116, 117, 118, 119, or 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about 100, 105, 110, 115, 120, 125, 130, 135, 140, 145, 150, 155, 160, 165, or 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about from 100 to 110 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about from 100 to 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about from 110 to 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about from 100 to 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about from 120 to 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about from 130 to 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 100 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 110 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 130 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 140 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 150 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 160 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 170 mg/mL at 25 degrees C.

In another embodiment, a liquid formulation of the present invention comprises at least about 15.00, 16.00, 17.00, 18.00, 19.00, 20.00, 21.00, 22.00, 23.00, 24.00, 25.00, 26.00, 27.00, 28.00, 29.00, 30.00, 31.00, 32.00, 33.00, 34.00, 35.00, 36.00, 37.00, 38.00, 39.00, 40.00, 41.00, 42.00, 43.00, 44.00, 45.00, 46.00, 47.00, 48.00, 49.00, 50.00, 51.00, 52.00, 53.00, 54.00, 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, 90.00, 91.00, 92.00, 93.00, 94.00, or 95.00 percent by weight of an omega-3 ester oil, at least about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.00, 2.00, 3.00, 4.00, 5.00, 6.00, 7.00, 8.00, 9.00, 10.00, 11.00, 12.00, 13.00, 14.00, 15.00, 16.00, 17.00, 18.00, 19.00, 20.00, 25.00, or 30.00 percent by weight C₁ to C₄ alcohol, and at least about 1.00, 2.00, 3.00, 4.00, 5.00, 6.00, 7.00, 8.00, 9.00, 10.00, 11.00, 12.00, 13.00, 14.00, 15.00, 16.00, 17.00, 18.00, 19.00, or 20.00 percent by weight of fenofibrate.

In another embodiment, a liquid formulation of the present invention comprises at least about 15.00, 16.00, 17.00, 18.00, 19.00, 20.00, 21.00, 22.00, 23.00, 24.00, 25.00, 26.00, 27.00, 28.00, 29.00, 30.00, 31.00, 32.00, 33.00, 34.00, 35.00, 36.00, 37.00, 38.00, 39.00, 40.00, 41.00, 42.00, 43.00, 44.00, 45.00, 46.00, 47.00, 48.00, 49.00, 50.00, 51.00, 52.00, 53.00, 54.00, 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, 90.00, 91.00, 92.00, 93.00, 94.00, or 95.00 percent by weight of an omega-3 ester oil, less than about 30.00, 25.00, 20.00, 15.00, 10.00, 5.00, or 2.50 percent by weight C₁ to C₄ alcohol, and at least about 1.00, 2.00, 3.00, 4.00, 5.00, 6.00, 7.00, 8.00, 9.00, 10.00, 11.00, 12.00, 13.00, 14.00, 15.00, 16.00, 17.00, 18.00, 19.00, or 20.00 percent by weight of fenofibrate.

In another embodiment, a medium-chain triglyceride such as a caprylic/capric triglyceride (e.g., Neobee® M5 Stepan Company) or a medium chain mono-diglyceride such as caprylic/capric mono-diglyceride (e.g., Capmul® MCM, Abitec Corporation) may be included in a formulation of the invention to facilitate digestion of the formulation or reduce the food effect. In another embodiment, a surfactant may be included in a formulation of the invention to enhance digestion of the formulation or reduce the food effect.

A surfactant-containing liquid formulation or medicament of the invention comprises a mixture of fenofibrate dissolved in a vehicle comprising an omega-3 ester or omega-3 alkyl ester and, optionally, a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant, and, optionally, (iv) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol; and
(b) the solubility of the fenofibrate in the vehicle is about 50, 60, 70, 80, 90, 100, 110, 120, 130, 140, 150, 160, 170, 180, 190, 200, 210, 220, 230, 240, 250, 260, 270, 280, 290, or 300 milligrams per milliliter at 25 degrees C.

In another embodiment, the surfactant-containing liquid formulation or medicament of the invention comprises a C₁ to C₄ alcohol, such as ethanol.

In an alternative embodiment, the surfactant increases the bioavailability of the non-aqueous formulation in the fasted state when compared with the non-aqueous formulation without surfactant.

A surfactant-containing liquid formulation or medicament of the invention comprises a mixture of fenofibrate dissolved in a vehicle comprising an omega-3 ester or omega-3 alkyl ester and, optionally, a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant, and, optionally, (iv) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol.

In another embodiment, the surfactant increases the solubility of the fenofibrate in the non-diluted liquid formulation.

In another embodiment of the invention, the surfactant is contained within the solid material of the capsule (i.e., within the gelatin casing or shell of a gelcap). In such an embodiment, the surfactant is prohibited from interacting with the omega-3 oil, the fenofibrate, and any other contents until after the solid capsule structure begins to dissolve (i.e., in vivo or in an aqueous environment).

In another embodiment, a liquid formulation according to the present invention comprises a surfactant with a weight percent less than about 50.00 percent of the total formulation. In another embodiment, a liquid formulation according to the present invention comprises a surfactant with a weight percent less than about 40.00 percent of the total formulation. In another embodiment, a liquid formulation according to the present invention comprises a surfactant with a weight percent less than about 30.00 percent of the total formulation. In another embodiment, a liquid formulation according to the present invention comprises a surfactant with a weight percent less than about 25.00 percent of the total formulation. In another embodiment, a liquid formulation according to the present invention comprises a surfactant with a weight percent less than about 20.00 percent of the total formulation. In another embodiment, a liquid formulation according to the present invention comprises a surfactant with a weight percent less than about 15.00 percent of the total formulation. In another embodiment, a liquid formulation according to the present invention comprises a surfactant with a weight percent less than about 10.00 percent of the total formulation. In another embodiment, a liquid formulation according to the present invention comprises a surfactant with a weight percent less than about 5.00 percent of the total formulation.

A formulation containing a high concentration of surfactant, according to the present invention, is one which has at least 30.00, 35.00, 40.00, 45.00, or 50.00 percent by weight of one or more surfactants. In another embodiment, a liquid formulation according to the present invention comprising a surfactant with a weight percent of about 25.00 or less, has a solubility of fenofibrate equal to or greater than that of formulations containing high concentrations of surfactant. In another embodiment, a liquid formulation according to the present invention comprising a surfactant with a weight percent of about 20.00 or less, has a solubility of fenofibrate equal to or greater than that of formulations containing high concentrations of surfactant. In another embodiment, a liquid formulation according to the present invention comprising a surfactant with a weight percent of about 15.00 or less, has a solubility of fenofibrate equal to or greater than that of formulations containing high concentrations of surfactant. In another embodiment, a liquid formulation according to the present invention comprising a surfactant with a weight percent of about 10.00 or less, has a solubility of fenofibrate equal to or greater than that of formulations containing high concentrations of surfactant. In another embodiment, a liquid formulation according to the present invention comprising a surfactant with a weight percent of about 5.00 or less, has a solubility of fenofibrate equal to or greater than that of formulations containing high concentrations of surfactant.

In another embodiment, the bioavailability of a liquid formulation of the invention is at least as high as that of the 160 mg dose of Tricor®. In one embodiment, a liquid formulation of the present invention which has about a 160 mg dose of fenofibrate per capsule has a bioavailability approximately equal to or higher than that of the 160 mg dose of Tricor®. In another embodiment, a liquid formulation of the present invention which has about a 150 mg dose of fenofibrate per capsule has a bioavailability approximately equal to that of the 160 mg dose of Tricor®. In another embodiment, a liquid formulation of the present invention which has about a 145 mg dose of fenofibrate per capsule has a bioavailability approximately equal to that of the 160 mg dose of Tricor®. In another embodiment, a liquid formulation of the present invention which has about a 140 mg dose of fenofibrate per capsule has a bioavailability approximately equal to that of the 160 mg dose of Tricor®. In another embodiment, a liquid formulation of the present invention which has about a 130 mg dose of fenofibrate per capsule has a bioavailability approximately equal to that of the 160 mg dose of Tricor®. In another embodiment, a liquid formulation of the present invention which has about a 120 mg dose of fenofibrate per capsule has a bioavailability approximately equal to that of the 160 mg dose of Tricor®.

A particular formulation of the invention comprises fenofibrate dissolved in a vehicle at a concentration of about 50, 60, 70, 80, 90, 100, 110, 120, 130, 140, 150, 160, 170, 180, 190, or 200 milligrams of fenofibrate per milliliter of formulation, wherein the vehicle consists of EPA and/or DHA ethyl esters, about 5.00, 6.00, 7.00, 8.00, 9.00, 10.00, 11.00, 12.00, 13.00, 14.00, or 15.00% by volume of ethanol, and a medium-chain triglyceride, and wherein the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of EPA and/or DHA ethyl esters, about 5.00, 6.00, 7.00, 8.00, 9.00, 10.00, 11.00, 12.00, 13.00, 14.00, or 15.00% by weight of ethanol, about 5.00, 6.00, 7.00, 8.00, 9.00, 10.00, 11.00, 12.00, 13.00, 14.00, or 15.00% by weight of the medium chain triglyceride, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate.

Another formulation of the invention comprises fenofibrate dissolved in a vehicle at a concentration of about 50, 60, 70, 80, 90, 100, 110, 120, 130, 140, 150, 160, 170, 180, 190, or 200 milligrams of fenofibrate per milliliter of formulation, wherein the vehicle comprises about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by volume of omega-3 ethyl esters, and about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and wherein: (1) the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl esters, about 5.00, 6.00, 7.00, 8.00, 9.00, 10.00, 11.00, 12.00, 13.00, 14.00, or 15.00% by weight of ethanol, and about 10.00, 11.00, 12.00, 13.00, 14.00, 15.00, 16.00, 17.00, 18.00, 19.00, or 20.00% by weight of fenofibrate, and (2) the molar ratio of unsaturated moieties contained with the omega-3 ethyl esters to the total moles of omega-3 ethyl ester is about 5 to about 6.

A particular capsule dosage form of the invention comprises fenofibrate relatively uniformly dispersed in a vehicle at a concentration of about 50, 60, 70, 80, 90, 100, 110, 120, 130, 140, 150, 160, 170, 180, 190, or 200 milligrams fenofibrate per milliliter of formulation, wherein the vehicle comprises about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by volume an omega-3 ethyl ester, and about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and wherein: (1) the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, or 15.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate, and (2) the molar ratio of unsaturated moieties contained with the omega-3 ethyl ester to the total moles of omega-3 ethyl ester is about 5 to about 6.

Another capsule dosage form of the invention comprises fenofibrate relatively uniformly dispersed in a vehicle at a concentration of about 50, 60, 70, 80, 90, 100, 110, 120, 130, 140, 150, 160, 170, 180, 190, or 200 milligrams fenofibrate per milliliter of formulation, wherein the vehicle comprises about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by volume EPA and/or DHA ethyl ester, and about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and wherein the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of EPA and/or DHA ethyl ester, about 5.00, 6.00, 7.00, 8.00, 9.00, or 10.00% by weight of ethanol, and about 10, 11.00, 12.00, 13.00, 14.00, 15.00, 16.00, 17.00, 18.00, 19.00, or 20.00% by weight of fenofibrate.

In another embodiment, a liquid formulation or medicament of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate completely solubilizes the fenofibrate at 25 degrees C.

In another embodiment, a liquid formulation or medicament of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 70 mg/mL at about 4 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 100 mg/mL at about 10 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 150 mg/mL at about 22 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 160 mg/mL at about 25 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 220 mg/mL at about 33 degrees C.

In another embodiment, a liquid formulation or medicament of the present invention comprising about 80.00, 81.00, 82.00, 83.00, 84.00, 85.00, 86.00, 87.00, 88.00, 89.00, 90.00, 91.00, 92.00, 93.00, 94.00, or 95.00% by weight of omega-3 ethyl ester and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 50 mg/mL at about 4 degrees C. In another embodiment, a liquid formulation of the present invention comprising about 80.00, 81.00, 82.00, 83.00, 84.00, 85.00, 86.00, 87.00, 88.00, 89.00, 90.00, 91.00, 92.00, 93.00, 94.00, or 95.00% by weight of omega-3 ethyl ester and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 100 mg/mL at about 22 degrees C. In another embodiment, a liquid formulation of the present invention comprising about 80.00, 81.00, 82.00, 83.00, 84.00, 85.00, 86.00, 87.00, 88.00, 89.00, 90.00, 91.00, 92.00, 93.00, 94.00, or 95.00% by weight of omega-3 ethyl ester and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 150 mg/mL at about 33 degrees C.

In another embodiment, a method of increasing the solubility of fenofibrate in a liquid formulation or a medicament containing an omega-3 oil is provided by adding from about 1 to about 25 percent by volume of an omega-3 ester-based oil. In one specific embodiment, the omega-3 oil exists as triglycerides. In another specific embodiment, the omega-3 oil exists as mono-diglycerides. In another specific embodiment, the omega-3 oil exists as free acids. In another specific embodiment, the omega-3 oil exists as phospholipids. In another specific embodiment, the omega-3 oil exists as a mixture of triglycerides, mono-diglycerides, and free acids. In another specific embodiment, the omega-3 oil exists as a mixture of triglycerides and mono-diglycerides. In another specific embodiment, the omega-3 oil exists as a mixture of triglycerides and free acids. In another specific embodiment, the omega-3 oil exists as a mixture of mono-diglycerides and free acids.

In another embodiment, a liquid formulation or medicament of the present invention can be stored for up to 8 weeks at about 25 degrees C. with no detectable degradation of fenofibrate. In another embodiment, a liquid formulation of the present invention can be stored for up to 12 weeks at about 25 degrees C. with no detectable degradation of fenofibrate. In another embodiment, a liquid formulation of the present invention can be stored for up to 16 weeks at about 25 degrees C. with no detectable degradation of fenofibrate.

In some formulations, it is possible for the fenofibrate, or a portion thereof, to precipitate out of solution during storage. This can be caused by, for example, a storage temperature significantly below room temperature. In another embodiment, a liquid formulation of the present invention further comprises pharmaceutically acceptable precipitation nuclei to promote the crystallization of multiple, small crystals. In another embodiment, a liquid formulation of the present invention is administered in slow-dissolving gelatin capsules, so as to increase the duration of time such capsules remain intact in the patient's stomach. For example, a slow-dissolving gelatin capsule can take 5, 6, 7, 8, 9, 10, 11, 12, 13, 14, 15, 16, 17, 18, 19, or 20 minutes or more to open in vivo. In another embodiment, a liquid formulation comprises pharmaceutically acceptable precipitation nuclei and is administered in slow-dissolving gelatin capsules. In another embodiment, a liquid formulation comprises pharmaceutically acceptable precipitation nuclei and is administered in slow-dissolving gelatin capsules so as to effectively provide completely solubilized fenofibrate upon capsule dissolution in vivo. In another embodiment, a liquid formulation of the present invention maintains fenofibrate in solution at a temperature of about 22 degrees C. In another embodiment, a liquid formulation of the present invention maintains fenofibrate in solution at a temperature of about 18 degrees C. In another embodiment, a liquid formulation of the present invention maintains fenofibrate in solution at a temperature of about 15 degrees C. In another embodiment, a liquid formulation of the present invention maintains fenofibrate in solution at a temperature of about 12 degrees C. In another embodiment, a liquid formulation of the present invention maintains fenofibrate in solution at a temperature of about 15 degrees C. and a fenofibrate concentration of at least 100 mg/mL. In another embodiment, a liquid formulation of the present invention maintains fenofibrate in solution at a temperature of about 15 degrees C. and a fenofibrate concentration of at least 110 mg/mL. In another embodiment, a liquid formulation of the present invention maintains fenofibrate in solution at a temperature of about 15 degrees C. and a fenofibrate concentration of at least 120 mg/mL. In another embodiment, a liquid formulation of the present invention maintains fenofibrate in solution at a temperature of about 15 degrees C. and a fenofibrate concentration of at least 130 mg/mL. In another embodiment, a liquid formulation of the present invention maintains fenofibrate in solution at a temperature of about 15 degrees C. and a fenofibrate concentration of at least 140 mg/mL. In another embodiment, a liquid formulation of the present invention maintains fenofibrate in solution at a temperature of about 15 degrees C. and a fenofibrate concentration of at least 150 mg/mL. In another embodiment, a liquid formulation of the present invention maintains fenofibrate in solution from its initial manufacture, through storage and handling, to administration.

In another embodiment, a method of preventing, reducing, and/or treating hypercholesterolemia, atherosclerosis, hypertriglyceridemia, cardiovascular events and disease including coronary events and cerebrovascular events, and coronary artery disease and/or cerebrovascular disease is provided. This method comprises administering an effective amount of a liquid formulation of the present invention to a mammal in need of such prevention, reduction, and/or treatment. In another embodiment, the mammal is a human.

The liquid formulations of the present invention can be prepared according to any one or more methods available in the art. For example, in one embodiment comprising omega-3 oil, fenofibrate, ethanol, and one or more surfactants, appropriate amounts of said formulation components can be mixed together at room temperature or at a slightly elevated temperature. Where one or more formulation components contain a solid which has precipitated from solution (e.g., a surfactant), such a component can be heated and mixed so as to induce resolubilization prior to combining with the remaining formulation components.

A therapeutically acceptable daily dosage of omega-3 oil has been recommended or considered via several national and international groups including, but not limited to, the American Heart Association (AHA) and the International Society for the Study of Fatty Acids and Lipids (ISSFAL). Table 1 includes daily dosage amounts of omega-3 as considered/recommended via several organizations.

TABLE 1
Daily dosages of omega-3

| Omega-3 dose | |
|---|---|
| (grams)/day | Comment |
| 0.65 | ISSFAL consideration (1999) |
| 1.0 | AHA recommended (2000, 2004) |
| 1.8 | Omacor ® dose |
| 3.0 | FDA limit on daily consumption, general population |
| 3.6 | Omacor ® dose |

In another embodiment, the present invention provides a novel polymorph of fenofibrate.

In another embodiment, the present invention provides a method of making a polymorph of fenofibrate, comprising:
(a) combining fenofibrate with one or more components so as to form a solution of fenofibrate;
(b) decreasing the temperature of said solution; and
(c) collecting a precipitated solid.

Liquid formulations of the invention may comprise any one polymorph of fenofibrate or a mixture of two or more polymorphs of fenofibrate. For example, a liquid formulation of the present invention may be prepared from fenofibrate (Form I), fenofibrate (Form II), or a mixture of Forms I and II.

Typical dosage forms of the invention comprise from about 10 mg to about 1000 mg, or an amount of from about 25 mg to about 500 mg, or an amount of from 40 mg to 400 mg, or an amount of from about 50 mg to about 200 mg of fenofibrate. For example, dosage forms comprising 30, 40, 50, 60, 70, 80, 90, 100, 110, 120, 130, 140, 145, 150, 160, 170, 180, 190, or 200 mg fenofibrate are included. More specifically, doses include 50, 100, 145, 150, and 160 mg of fenofibrate.

Liquid formulations of the present invention, optionally, can be administered in soft gelatin capsules. Such soft gelatin capsules can be in any shape, for example, oval or oblongs. The volume of such capsules can be between about 0.5 mL and about 1.5 mL. For example, about 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, or 1.50 mL. In one embodiment, one dose consists of a single capsule. In another embodiment, one dose consists of two capsules. In another embodiment, one dose consists of three or more capsules. Optionally, each dose can be packaged individually in a blister-pack. In another embodiment, the soft gelatin material is both chemically and physically stable while in contact with a liquid formulation of the invention. In another embodiment, the soft gelatin material prevents the alcohol in the liquid formulation from escaping the capsule. In another embodiment, the soft gelatin material prevents a significant amount of the alcohol in the liquid formulation from escaping the capsule.

All aforementioned ranges (e.g., 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00) of percent identity are to be taken as including, and providing written description and support for, any fractional percentage, in intervals of 0.01%.

It is generally practiced that the process for preparing the formulations include the use of a purge of an inert gas. Such inert gases are for example, nitrogen, argon, and the like. The use of an isolator to maintain low oxygen conditions is desirable, but not required for storage of the present formulation.

These and other embodiments of the invention are illustrated further in the following examples, which are illustrative and in no way limiting.

### EXEMPLIFICATION

### Materials and Methods

### Powder X-Ray Diffraction

All X-ray powder diffraction patters were obtained using a D/Max Rapid X-ray Diffractometer (Rigaku/MSC, The Woodlands, TX, U.S.A.) equipped with a copper source (Cu/K_{α}1.5406 Å), manual x-y stage, and 0.3 mm collimator. A sample was loaded into a 0.3 mm quartz capillary tube (Charles Supper Company, Natick, Mass., U.S.A.) by sectioning off the closed end of the tube and tapping the small, open end of the capillary tube into a bed of the powdered sample or into the sediment of a slurried sample. The precipitate can be amorphous or crystalline. The loaded capillary tube was mounted in a holder that was placed and fitted into the x-y stage. A diffractogram was acquired using control software (RINT Rapid Control Software, Rigaku Rapid/XRD, version 1.0.0 (©1999 Rigaku Co.)) under ambient conditions at a power setting of 46 kV at 40 mA in transmission mode, while oscillating about the omega-axis from 0-5 degrees at 1 degree/second, and spinning about the phi-axis over 360 degrees at 2 degrees/second. The exposure time was 15 minutes unless otherwise specified.

The diffractogram obtained was integrated of 2-theta from 2-60 degrees and chi (1 segment) from 0-36 degrees at a step size of 0.02 degrees using the cyllnt utility in the RINT Rapid display software (RINT Rapid display software, version 1.18 (Rigaku/MSC)) provided by Rigaku with the instrument. The dark counts value was set to 8 as per the system calibration by Rigaku. No normalization or omega, chi or phi offsets were used for the integration.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, or by about +/−0.05. The entire pattern or most of the pattern peaks may also shift by about +/−0.1 degrees to about +/−0.2 degrees due to differences in calibration, settings, and other variations from instrument to instrument and from operator to operator. All reported PXRD peaks in the Figures, Examples, and elsewhere herein are reported with an error of about ±0.1 degrees 2-theta. Unless otherwise noted, all diffractograms are obtained at about room temperature (about 24 degrees C. to about 25 degrees C.).

Unless otherwise specified, the term fenofibrate refers to fenofibrate Form I (the currently marketed form) in the Exemplification.

### Solubility Measurements Via Ultraviolet (UV) Absorption

First, a calibration curve was constructed by preparing known concentrations of fenofibrate in absolute ethanol in volumetric flasks. At each concentration, 200 microliters of the solution was transferred into a 96-well clear bottom UV plate. The sample absorbance was measured at 280 nm (unless otherwise noted) in a UV spectrophotometer. It was found that the absorbance vs. concentration correlation was linear to at least 100 micrograms/mL.

To measure the fenofibrate concentration in the sample, a small aliquot was taken and diluted (typically 2000-fold) with absolute ethanol in a volumetric flask to a final approximate concentration of less than 100 micrograms/mL. The absorbance at 280 nm (unless otherwise noted) is measured and the solubility is calculated based on the calibration curve.

### Example 1

### Solubility of Fenofibrate in Different Liquid Vehicles

Saturated solutions of fenofibrate in various liquid vehicles were prepared in 1.5 mL glass vials by stepwise addition of fenofibrate powder to approximately 0.5-1 mL of liquid vehicle. If the powder dissolved completely, more fenofibrate was added until an excess of powder was observed. The samples were then stirred overnight at 25° C. controlled temperature before being filtered through a 0.2 micrometer PVDF syringe filter. The filtrate was diluted with n-heptane and analyzed via normal phase HPLC.

Table 2 summarizes the solubility of Fenofibrate in various liquid vehicles.

TABLE 2
Solubility of fenofibrate in various liquid vehicles

| | | Solubility (mg/ml, |
|---|---|---|
| No. | Mixture | at 25 degrees C.) |
| 1 | 100% E9501EE* | 107 |
| 2 | 100% Ethanol | 57 |
| 3 | 100% Omegabrite | 113 |
| 4 | 100% Myvacet 9-45 | 115 |
| 5 | 100% Epax 1050TG | 76 |
| 6 | 100% Epax 4510TG | 80 |
| 7 | 100% Cod liver oil | 52 |
| 8 | 100% Natural fish oil | 55 |
| 9 | 100% Flaxseed oil | 57 |
| 10 | 100% Flax-borage | 59 |

*E9501EE comprises 95 percent EPA, 1 percent DHA, as ethyl esters (mass percent)

Based on available composition data, Table 3 below compares fenofibrate solubility and omega-3 content in different vehicles.

TABLE 3
Fenofibrate solubility and omega-3 content (mass percent) in various
vehicles

| | | | | | Solubility |
|---|---|---|---|---|---|
| | | DHA | Other | Total | (mg/mL) |
| | EPA % | % | omega-3 % | % | at 25 deg C. |
| Cod Liver Oil | 11 | 11 | 0 | 22 | 52 |
| Natural Fish Oil | 18 | 12 | 0 | 30 | 55 |
| Flax Seed Oil | 0 | 0 | 50 | 50 | 57 |
| Flax-Borage Oil | 7 | 5 | 50 | 62 | 59 |
| EPAX ® 1050 TG | 10 | 50 | 0 | 60 | 76 |
| EPAX ® 4510 TG | 45 | 10 | 0 | 55 | 80 |
| E9501EE* | 95 | 1 | 0 | 96 | 107 |
| Omegabrite ® | 75 | 11 | 6 | 92 | 113 |

*E9501EE comprises 95 percent EPA, 1 percent DHA (mass percent) as ethyl esters

It is believed that, among other factors, fenofibrate solubility in omega-3 oils may also be proportional to the number of double-bonds present in the vehicle.

### Example 2

### Equilibrium Fenofibrate Solubility in Ethyl Esters Versus Triglycerides

Table 4 shows a comparison of fenofibrate solubility in ethyl esters and that in corresponding triglycerides at 25 degrees C. Polarity and the number of C═C double bonds correlate with increased fenofibrate solubility. Importantly, fenofibrate shows higher solubility consistently in ethyl esters than in a corresponding triglyceride.

TABLE 4
Fenofibrate solubility at 25 degrees C. in ethyl esters and in
triglycerides

| Vehicle | Description | Solubility (mg/mL) |
|---|---|---|
| Ethyl caprylate | C8, ethyl ester | 177.8 |
| Ethyl caprate | C10, ethyl ester | 142.2 |
| Neobee M5 | C8 and C10, triglyceride | 82.0 |
| Ethyl oleate | C18, 1 double bond, ethyl ester | 86.7 |
| Triolein | C18, 1 double bond, triglyceride | 48.9 |
| Ethyl linoleate | C18, 2 double bonds, ethyl ester | 92.3 |
| Trilinolein | C18, 2 double bonds, | 61.2 |
| | triglyceride | |

### Example 3

### Determination of Increased Solubilization Power with Ethanol and Ethyl Esters

A saturated solution of fenofibrate (125.80 mg) in TG361724 fish oil was prepared by adding the fish oil to the fenofibrate up to a volume of 1 mL. The fish oil was comprised of triglycerides. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure TG361724 is reported below in Table 5.

A saturated solution of fenofibrate (145.47 mg) in a 90:10 solution by volume of TG361724:ethanol was prepared by adding the fish oil:ethanol mixture to the fenofibrate up to a volume of 1 mL. The fish oil was comprised of triglycerides. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 TG361724:ethanol is reported below in Table 5.

A saturated solution of fenofibrate (125.46 mg) in E351923 was prepared by adding the fish oil to the fenofibrate up to a volume of 1 mL. The fish oil was comprised of ethyl esters. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure E351923 is reported below in Table 5.

A saturated solution of fenofibrate (201.74 mg) in a 90:10 solution by volume of E351923:ethanol was prepared by adding the fish oil:ethanol mixture to the fenofibrate up to a volume of 1 mL. The fish oil was comprised of ethyl esters. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 E351923:ethanol is reported below in Table 5.

TABLE 5
Fenofibrate solubility in several oils and oil:ethanol mixtures
at 25 degrees C.

| Liquid Vehicle | Solubility (mg/mL) |
|---|---|
| TG361724 | 67.3 |
| 90:10 TG361724:ethanol | 88.5 |
| E351923 | 95.6 |
| 90:10 E351923:ethanol | 129.0 |

A saturated solution of fenofibrate (130.9 mg) in E107104 was prepared by adding the fish oil to the fenofibrate up to a volume of 1 mL. The fish oil was rich in DHA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure E107104 is reported below in Table 6.

A saturated solution of fenofibrate (151.3 mg) in a 95:5 solution by volume of E107104:ethanol was prepared by adding the fish oil:ethanol mixture to the fenofibrate up to a volume of 1 mL. The fish oil was rich in DHA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 95:5 E107104:ethanol is reported below in Table 6.

A saturated solution of fenofibrate (161.6 mg) in a 90:10 solution by volume of E107104:ethanol was prepared by adding the fish oil:ethanol mixture to the fenofibrate up to a volume of 1 mL. The fish oil was rich in DHA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 E107104:ethanol is reported below in Table 6.

A saturated solution of fenofibrate (154.2 mg) in E970002 was prepared by adding the fish oil to the fenofibrate up to a volume of 1 mL. The fish oil was rich in EPA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure E970002 is reported below in Table 6.

A saturated solution of fenofibrate (204.8 mg) in a 90:10 solution by volume of E970002:ethanol was prepared by adding the fish oil:ethanol mixture to the fenofibrate up to a volume of 1 mL. The fish oil was rich in EPA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 E970002:ethanol is reported below in Table 6.

TABLE 6
Fenofibrate solubility in several EPA and DHA-rich oils and
oil:ethanol mixtures at 25 degrees C.

| Liquid Vehicle | Solubility (mg/mL) |
|---|---|
| E107104 | 102.2 |
| 95:5 E107104:ethanol | 124.5 |
| 90:10 E107104:ethanol | 132.1 |
| E970002 | 106.7 |
| 90:10 E970002:ethanol | 140.8 |

Table 5 shows an increased solubility of fenofibrate in omega-3 oils when ethanol is added to the formulation. Although this increase is seen in omega-3 triglyceride-based oils as well as omega-3 ethyl ester-based oils, it is only the ethyl ester-based omega-3 oils that provide the fenofibrate solubility at and above 100 mg/mL which is necessary for liquid formulations of the present invention. Table 6 shows a similar increase in fenofibrate solubility with the addition of ethanol. Also, omega-3 oils with a high content of DHA and omega-3 oils with a high content of EPA both provide similar solubilization power. Based on the above data, the ratio of EPA:DHA does not appear to be a critical variable for the increased solubilization power of fenofibrate in omega-3 oil.

### Example 4

### Fenofibrate Polymorph (Form II)

In a 5 mL glass vial, 500.3 mg of Gelucire® 44/14 and 500.3 mg of poloxamer 407 were dispensed and continuously mixed with a magnetic stir-bar. The mixture was heated in a water bath to 85 degrees C. until all components were molten. 670.1 mg of fenofibrate was slowly added and mixed for an additional 20 minutes. The temperature was reduced to 70 degrees C. and mixed for another 20 minutes. 50 microliters of this sample was collected and placed into a glass vial that had been prepared at 70 degrees C. The glass vial was immediately cooled by placement into an acetone/dry ice bath and then placed at 4 degrees C. A solid formed and was collected for PXRD analysis. The solid was determined to be a fenofibrate polymorph (Form II).

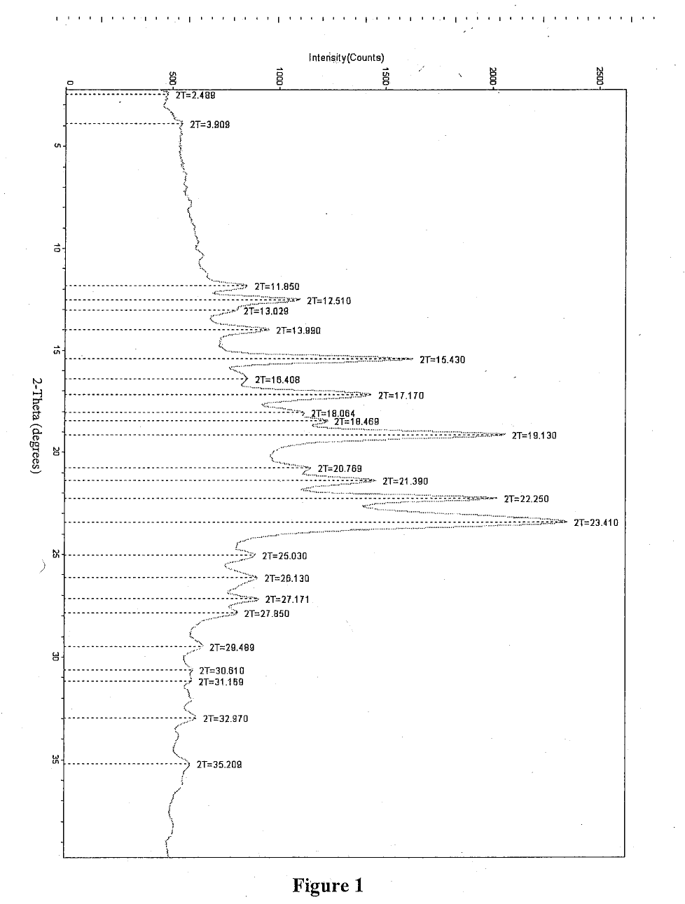

Crystals representative of those obtained by completing the method above were characterized using PXRD (See FIG. 1). The fenofibrate polymorph (Form II) exhibits a PXRD diffractogram comprising peaks, for example, at about 11.85, 12.51, 13.99, 15.43, 17.17, 18.47, 19.13, 21.39, 22.25, 23.41, 25.03, 26.13, and 27.17 degrees 2-theta (Rigaku, data as collected).

The fenofibrate polymorph (Form II) was also prepared in pure Gelucire® 44/14 and in pure poloxamer 407.

### Example 5

### Pharmacokinetic Analysis of Fenofibric Acid in Humans

The pharmacokinetics of fenofibric acid were evaluated in humans. 18 healthy subjects (male and female) were selected for this study. The study design was a single-dose, 3 treatment, and 3-sequence, 3-period crossover with a washout interval of at least one-week between each period. An equal number of subjects (i.e. six) was randomly assigned to each of the three sequences. Following an overnight fast of at least 10 hours, subjects were given a single dose of the following test or reference treatment with 240 mL of water:

1. Fenofibrate/omega-3, 160 mg capsule after a standard breakfast;
2. Fenofibrate/omega-3, 160 mg capsule after an overnight fast; and
3. Tricor®, 160 mg tablet after a standard breakfast;
wherein the fenofibrate/omega-3 administered formulation comprised the components and amounts shown in Table 7.

TABLE 7
Fenofibrate/omega-3 Formulation Administered to Humans

| Component | Weight percent | Per dose (mg) (2 capsules) |
|---|---|---|
| Fenofibrate | 15.11 | 160.00 |
| E681010 | 73.69 | 780.16 |
| Ethanol | 11.20 | 118.57 |

Venous blood samples were collected pre-dose (0 hours) and 1, 2, 3, 4, 6, 8, 10, 14, 24, 34, 48 and 72 hours post-dose. Plasma from the collected blood samples were promptly separated and frozen until assayed using a validated assay for fenofibric acid in human plasma with a lower limit of quantitation of 20.1 ng/mL.

The pharmacokinetic measures, including AUC₀₋ₜ, AUC_{0-inf}, Cₘₐₓ, Tₘₐₓ and t½ were calculated from the individual concentration-time data for fenofibric acid using PhAST software (Phoenix international). Analysis of variance (ANOVA) was performed for log-transformed data of AUC₀₋ₜ, AUC_{0-inf}, and Cₘₐₓ. In Table 8 below, “Cₘₐₓ” is the maximum blood plasma concentration, “AUC₀₋ₜ” is the area under the curve from time point 0 to 72 hours post-dose, “AUC_{0-inf}” is the extrapolated area under the curve, “t_{1/2}” is the amount of time for the blood plasma level to decrease to half of the Cₘₐₓ level beginning at administration, “Tₘₐₓ” is the time to maximum blood plasma concentration from administration, and “F” is the percent bioavailability.

TABLE 8
Summary of Mean (SD) Pharmacokinetic Parameters of Fenofibric Acid in Humans Following
Oral Administration of Two Formulations of Fenofibrate

| | AUC₀₋ₜ | AUC_{0-inf} | Cmax | Half-life | Tmax | F | F |
|---|---|---|---|---|---|---|---|
| Treatment | (ng/mL × hr) | (ng/mL × hr) | (ng/mL) | (hr) | (hr) | (AUC₀₋ₜ) | (AUC_{0-inf}) |
| Tricor 160 mg fed | 167728 | 182132 | 9899 | 19.2 | 5.94 | NA | NA |
| | (43953.8) | (55371.7) | (3075.1) | (5.65) | (7.62) | | |
| Fenofibrate/Omega- | 175534 | 189497 | 8719 | 17.7 | 10.0 | 105.0 | 104.1 |
| 3 160 mg fed | (51051.4) | (60123.0) | (2438.4) | (4.85) | (7.06) | (20.6) | (16.1) |
| Fenofibrate/omega-3 | 110032^{a} | 166326 | 3559^{a} | 37.1 | 15.8 | 67.2 | 87.9 |
| 160 mg fasted | (58220.0) | (104979.1) | (3421.4) | (29.2) | (12.73) | (37.2) | (39.4) |

^{a}Statistically significant difference (p < 0.05) compared to Tricor ®

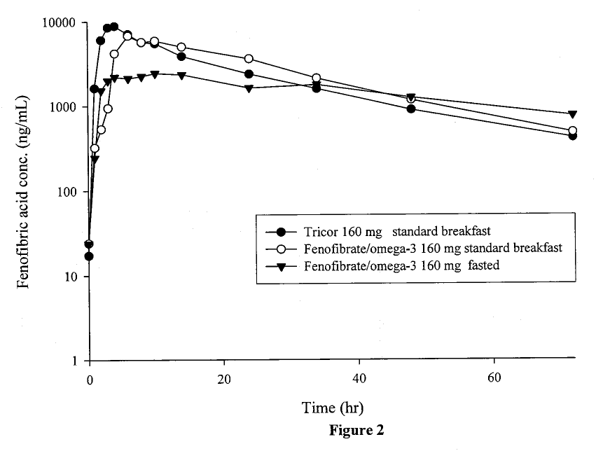

FIG. 2 shows a semi-log plot of mean plasma concentration of fenofibric acid in humans following oral administration of two formulations of fenofibrate.

### Example 6

### Fenofibrate Solubility in Various Oils at 15 Degrees C.

Table 9 shows the solubility of fenofibrate measured at 15 degrees C. in several oils and in several oil/ethanol mixtures.

TABLE 9
Solubility of fenofibrate in several vehicles at 15 degrees C.

| | Solubility |
|---|---|
| Vehicle | mg/mL |
| 85/15 wt % Captex ® 200/Ethanol | 126 |
| Captex ® 200 | 93 |
| 50/50 wt % Myvacet ® 9-45K/E681010 | 92 |
| E681010 | 90 |
| Myvacet ® 9-45K | 90 |
| 85/15 wt % Crodamol EO/Ethanol | 89 |
| Triomega ® Omega-3 | 85 |
| 85/15 wt % Eumulgin ® 05/Ethanol | 69 |
| 85/15 wt % Oleic Acid/Ethanol | 65 |
| Crodamol EO | 64 |
| 85/15 wt % Campul ® MCM/Ethanol | 63 |
| Epax ® 4510TG | 61 |
| Epax ® 1050TG | 60 |
| 85/15 wt % Peceol/Ethanol | 51 |
| FlaxSeed Oil | 47 |
| Cod Liver Oil | 43 |
| Oleic Acid | 39 |

In the table above and throughout the disclosure, Captex® 200 is also known as propylene glycol dicaprylate/dicaprate, Myvacet® 9-45K is also known as acetylated monoglycerides, Crodamol EO is also known as ethyl oleate, Capmul® MCM is also known as capric/caprylic glycerides, Peceol is also known as glycerol oleate, Epax® 4510TG is a concentrate containing 45 percent EPA and 10 percent DHA (triglycerides), Epax® 1050TG is a concentrate containing 10 percent EPA and 50 percent DHA (triglycerides), and Eumulgin® 05 is also known as ethocylated oleyl cetyl alcohol.

### Example 7

### Fenofibrate Solubility as a Function of Ethanol Concentration

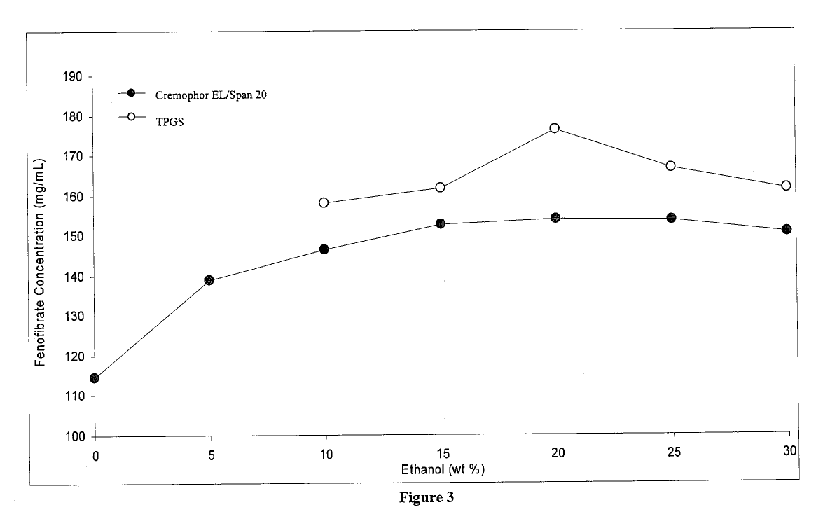

The solubility of fenofibrate was studied in two surfactant-containing formulations as a function of ethanol concentration. Formulation one comprised E681010:ethanol:Cremophor EL:Span 20, wherein the weight percent of Cremophor EL and Span 20 were each maintained at 10 percent. (For example, the samples contained component weight ratios of 80:0:10:10, 75:5:10:10, 70:10:10:10, 65:15:10:10, 60:20:10:10, 55:25:10:10, and 50:30:10:10.) Note, Span 20 is also known as sorbitan monolaurate. Formulation two contained E681010:ethanol:TPGS, wherein the weight percent of TPGS was maintained at 20 percent. (For example, the samples contained component weight ratios of 70:10:20, 65:15:20, 60:20:20, 55:25:20, and 50:30:20.) Note, TPGS is also known as d-alpha-tocopheryl polyethylene glycol 1000 succinate. FIG. 3 shows the data from zero percent to 30 percent ethanol by weight.

### Example 8

### Fenofibrate Solubility as a Function of Temperature

Formulation components were weighed and mixed to form homogeneous solutions. Excess fenofibrate was added to 1 mL of the premixed formulation into 10 mL vials. A stir bar was added and the vials were crimped. The formulations were incubated at fixed temperatures (e.g., 15, 25, 32° C.) using a circulating water bath for 24 to 72 hours under constant mixing. Post incubation, 1 mL of each mixture was filtered via syringe with a 0.45 micrometer pore size, 13 mm, PTFE filter. 50 to 100 microliters of the filtered solution was collected and diluted 1000-fold in volumetric flask with 30/70 v/v acetonitrile-water. Diluted samples were analyzed for fenofibrate content using HPLC with UV detection.

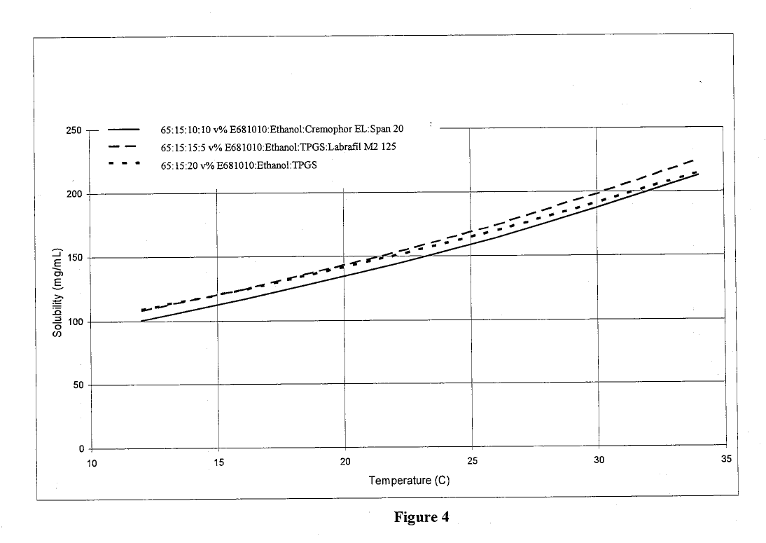

FIG. 4 shows the temperature dependence of fenofibrate solubility for three different formulations. The first formulation comprises E681010:ethanol:Cremophor EL:Span 20 in a ratio of 65:15:10:10. The second formulation comprises E681010:ethanol:TPGS:Labrafil M2125 in a ratio of 65:15:15:5. The third formulation comprises E681010:ethanol:TPGS in a ratio of 65:15:20. Note, Labrafil M2125 is also known as linoleoyl polyoxylglycerides.

### Example 9

### Characterization of Emulsification Behavior

Filtered samples from the solubility studies were also used to characterize the emulsification behavior of several formulations. Fenofibrate was saturated in these samples. In the study, 64 microliters of formulation was added to a 20 mL solution of 34.2 mM sodium chloride in deionized water. (This simulates the addition of 0.8 mL gelatin capsule to a 250 mL resting stomach volume of fluid.) The sodium chloride solution represents simulated gastric fluid in the absence of a surfactant wetting agent. Observations to the emulsification process were: 1) Degree of Emulsification (in order of decreasing degrees): microemulsion, coarse emulsion, partial emulsion, poor emulsion, or no emulsification (none); and 2) Dispersion Speed: fast or slow.

Table 10 shows several surfactant-containing formulations of fenofibrate in E681010 and ethanol with variable ratios of oil, ethanol, and surfactant. The solubility measurement described in Table 10 were taken at 27 degrees C. The emulsification classification was completed at 37 degrees C. Note: All reports of weight percent in Table 10 are rounded to the nearest whole number, and therefore may include approximations of up to +/−0.5 percent by weight.

TABLE 10
Fenofibrate Solublity and Emulsification Data

| | Average Solubility | Degree of | Dispersion |
|---|---|---|---|
| Formulation Composition | (mg/mL) | Emulsification | Speed |
| 100 wt % E681010 | 126 | None | N/A |
| 89/11 wt % E681010/Ethanol | 180 | None | N/A |
| 66/12/22 wt % E681010/Ethanol/Cremophor EL | 158 | Poor | Fast |
| 52/8/21/19 wt % E681010/Ethanol/Cremophor EL/Span20 | 139 | Coarse | Fast |
| 65/12/11/9/3 wt % E681010/Ethanol/Cremophor EL/ | 153 | None | N/A |
| Span20/Ethanolamine | | | |
| 67/13/11/10 wt % E681010/Ethanol/CremophorEL/ | 167 | Coarse | Fast |
| Crill 1 NF | | | |
| 70/11/9/10 wt % E681010/Ethanol/Cremophor EL/ | 174 | Poor | Slow |
| Alcolec EM | | | |
| 67/13/11/10 wt % | 161 | Coarse | Fast |
| E681010/Ethanol/CremophorEL/Span20 | | | |
| 66/13/11/9 wt % E681010/Ethanol/CremophorEL/Span80 | 154 | Partial | Fast |
| 66/13/11/10 wt % E681010/Ethanol/CremophorEL/ | 151 | Poor | Slow |
| Labrafil M2125 CS | | | |
| 67/12/11/10 wt % | 156 | Poor | Slow |
| E681010/Ethanol/CremophorEL/Labrasol | | | |
| 66/12/10/12 wt % | 149 | Poor | Slow |
| E681010/Ethanol/CremophorEL/Polysorbate 20 | | | |
| 65/16/15/4 wt % E681010/Ethanol/Cremophor EL/ | 168 | Partial | Fast |
| Span20 | | | |
| 66/13/16/6 wt % E681010/Ethanol/CremophorEL/Span80 | 148 | DS | Fast |
| 67/13/15/5 wt % E681010/Ethanol/CremophorEL/ | 145 | Poor | Fast |
| Labrafil M2125 CS | | | |
| 67/13/15/6 wt % E681010/Ethanol/Cremophor/Labrasol | 147 | Poor | Fast |
| 67/13/2/19 wt % E681010/Ethanol/Cremophor EL/ | 169 | None | N/A |
| Span 20 | | | |
| 67/12/6/15 wt % E681010/Ethanol/Cremophor EL/ | 167 | Partial | Fast |
| Span 20 | | | |
| 67/13/6/15 wt % E681010/Ethanol/CremophorEL/ | 151 | Poor | Fast |
| Labrafil M2125 CS | | | |
| 66/12/6/16 wt % | 152 | None | N/A |
| E681010/Ethanol/CremophorEL/Labrasol | | | |
| 66/12/6/15 wt % | 147 | Poor | Slow |
| E681010/Ethanol/CremophorEL/Polysorbate 20 | | | |
| 66/13/11/10/1 wt % | 166 | Partial | Fast |
| E681010/Ethanol/CremophorEL/Span20/GlycocholicAcid | | | |
| 69/13/16/2 w % E681010/Ethanol/CremophorEL/ | 143 | Partial | Slow |
| Glycocholic Acid | | | |
| 66/8/26 wt % E681010/Ethanol/TPGS | 145 | Partial | Slow |
| 66/13/21 wt % E681010/Ethanol/TPGS | 164 | Partial | Slow |
| 76/8/16 wt % E681010/Ethanol/TPGS | 158 | Poor | Slow |
| 77/12/10 wt % E681010/Ethanol/TPGS | 178 | Poor | Fast |
| 76/4/20 wt % E681010/Ethanol/TPGS | 138 | Poor | Slow |
| 61/8/25/5 wt % E681010/Ethanol/TPGS/Labrafil M2125 | 167 | N/A | N/A |
| 66/8/21/5 wt % E681010/Ethanol/TPGS/Labrafil M2125 | 162 | Partial | Slow |
| 68/10/13/10 wt % E681010/Ethanol/TPGS/Poloxamer331 | 162 | None | N/A |
| 68/13/10/9 wt % E681010/Ethanol/TPGS/Span80 | 158 | Poor | Slow |
| 67/13/16/5 wt % E681010/Ethanol/TPGS/Labrafil M2125 | 174 | N/A | N/A |
| 68/12/15/5 wt % E681010/Ethanol/TPGS/ | 157 | Poor | Slow |
| Labrafil M1944CS | | | |
| 67/13/16/5 wt % E681010/Ethanol/TPGS/ | 175 | Partial | Fast |
| Labrafil M2125CS | | | |
| 66/13/16/4 wt % E681010/Ethanol/TPGS/Span80 | 157 | Poor | Slow |
| 67/13/17/4 wt % E681010/Ethanol/TPGS/Span85 | 161 | Poor | Fast |
| 66/13/19/3 wt % E681010/Ethanol/TPGS/ | 175 | Poor | Fast to |
| Labrafil M2125CS | | | Slow |
| 65/22/12 wt % E681010/Ethanol/Tween80 | 170 | Poor | Fast |
| 77/11/13 wt % E681010/Ethanol/Tween80 | 176 | None | N/A |
| 72/5/11/12 wt % E681010/Ethanol/Tween80/Span85 | 167 | None | N/A |
| 66/21/12/wt % E681010/Ethanol/Tween85 | 165 | Poor | Fast |
| 77/10/13 wt % E681010/Ethanol/Tween85 | 176 | None | N/A |

### Example 10

### Surfactant-Containing Fenofibrate Formulations

Two formulations were prepared for the administration of 145 mg of fenofibrate. Formulation A contained 145 mg fenofibrate in an 800 microliter capsule (91 mg/mL fenofibrate).

TABLE 11
Formulation A

| | | Per dose (mg)- |
|---|---|---|
| Component | Weight Percent | 2 (capsules) |
| Fenofibrate | 10.6 | 145 |
| E681010 | 58.1 | 794 |
| Ethanol | 13.4 | 183 |
| Cremophor EL | 8.9 | 122 |
| Span 20 | 8.9 | 122 |

Formulation B contained 145 mg fenofibrate in an 650 microliter capsule (111 mg/mL fenofibrate).

TABLE 12
Formulation B

| | | Per dose (mg)- |
|---|---|---|
| Component | Weight Percent | (2 capsules) |
| Fenofibrate | 12.6 | 145 |
| E681010 | 56.8 | 656 |
| Ethanol | 13.2 | 152 |
| Cremophor EL | 8.7 | 101 |
| Span 20 | 8.7 | 101 |

Two formulations were also prepared for the administration of 130 mg of fenofibrate. Formulation C contained 130 mg fenofibrate in an 800 microliter capsule (81 mg/mL fenofibrate).

TABLE 13
Formulation C

| | | Per dose (mg)- |
|---|---|---|
| Component | Weight Percent | (2 capsules) |
| Fenofibrate | 9.6 | 130 |
| E681010 | 58.8 | 800 |
| Ethanol | 13.6 | 185 |
| Cremophor EL | 9.0 | 123 |
| Span 20 | 9.0 | 123 |

Formulation D contained 130 mg fenofibrate in an 650 microliter capsule (100 mg/mL fenofibrate).

TABLE 14
Formulation D

| | | Per dose (mg)- |
|---|---|---|
| Component | Weight Percent | (2 capsules) |
| Fenofibrate | 11.5 | 130 |
| E681010 | 57.5 | 648 |
| Ethanol | 13.2 | 149 |
| Cremophor EL | 8.9 | 100 |
| Span 20 | 8.9 | 100 |

### Example 11

### Physical Stability Characterization of Fenofibrate in Various Oils at 15 Degrees C.

Fenofibrate solutions were prepared at a concentration of 65 mg/mL in pure oil and mixtures of oil and ethanol at room temperature. The solutions were incubated at 15 degrees C. and periodically observed for precipitation of fenofibrate.

Table 15 shows results of visual observation of oil samples with 13 percent w/w ethanol after 18 days at 15 degrees C.

TABLE 15
Physical Stability of Fenofibrate in Mixtures of Oil and Ethanol

| Oil with 13 wt % Ethanol | 18-day observation |
|---|---|
| Myvacet | Clear solution |
| Epax 1050TG | Clear solution |
| Epax 4510TG | Clear solution |
| Cod Liver Oil | Clear solution |
| E681010 | Clear solution |

Table 16 shows results of visual observation of pure oil samples after 18 days at 15 degrees C.

TABLE 16
Physical Stability of Fenofibrate in Pure Oil

| Oil | 18-day observation |
|---|---|
| Myvacet | Clear solution |
| Epax 1050TG | Precipitation |
| Epax 4510TG | Precipitation |
| Cod Liver Oil | Precipitation* |
| E681010 | Clear solution |

*= This sample did not form a 65 mg/mL solution at room temperature.

As suggested in the data above, ethanol appears to enhance the physical stability of fenofibrate solubilized in some oils.

### Example 12

### Fenofibrate Formulations

The following formulations comprise fenofibrate in about 145 mg doses, where two capsules are administered per dose. Table 17 describes several embodiments of non-surfactant-containing fenofibrate formulations.

TABLE 17
Fenofibrate formulations without surfactant

| | Fenofibrate Concentration | | |
|---|---|---|---|
| | (mg/mL) | | |
| | 80 | 90 | 100 |
| Formulation Density (g/mL) | 0.912 | 0.918 | 0.914 |
| Dose Volume (mL) | 1.81 | 1.61 | 1.45 |
| Capsule Volume (mL) | 0.907 | 0.805 | 0.725 |
| E681010 dose (g) | 1.31 | 1.16 | 1.02 |
| DHA and EPA content (g) | 1.02 | 0.90 | 0.80 |
| Total Omega-3 content (g) | 1.14 | 1.01 | 0.89 |
| Composition - Mass Percent | | | |
| Fenofibrate | 8.8 | 9.8 | 10.9 |
| E681010 | 79.2 | 78.3 | 77.3 |
| Ethanol | 12.0 | 11.9 | 11.8 |

Table 18 describes several embodiments of surfactant-containing fenofibrate formulations.

TABLE 18
Fenofibrate formulations comprising surfactant

| | Fenofibrate Concentration | | |
|---|---|---|---|
| | (mg/mL) | | |
| | 70 | 80 | 90 |
| Formulation Density (g/mL) | 0.938 | 0.935 | 0.943 |
| Dose Volume (mL) | 2.07 | 1.81 | 1.61 |
| Capsule Volume (mL) | 1.033 | 0.906 | 0.806 |
| E681010 dose (g) | 1.17 | 1.01 | 0.89 |
| DHA and EPA content (g) | 0.91 | 0.78 | 0.69 |
| Total Omega-3 content (g) | 1.02 | 0.88 | 0.78 |
| Composition - Mass Percent | | | |
| Fenofibrate | 7.5 | 8.6 | 9.5 |
| E681010 | 60.1 | 59.4 | 58.8 |
| Ethanol | 13.9 | 13.7 | 13.6 |
| Cremophor EL | 9.2 | 9.1 | 9.0 |
| Span 20 | 9.3 | 9.2 | 9.1 |

Table 19 describes a fenofibrate formulation where the solubility of fenofibrate is 106 mg/mL at 15 degrees C. The actual fenofibrate concentration in the formulation is 90.6 mg/mL. A single dose of this formulation (two capsules) includes 0.83 grams of omega-3 oil. This formulation provides similar emulsification to that observed in a similar formulation with a greater percentage of ethanol (13.6 weight percent).

TABLE 19
Fenofibrate formulation comprising surfactant

| Component | Weight Percent | Per Dose (mg) (2 capsules) |
|---|---|---|
| Fenofibrate | 9.6 | 145.0 |
| E681010 | 63.4 | 957.6 |
| Ethanol | 9.0 | 135.9 |
| Cremophor EL | 9.0 | 135.9 |
| Span 20 | 9.0 | 135.9 |

### Example 13

### Solubility Studies at 4 and 15 Degrees C.

Table 20 includes fenofibrate solubility data of four liquid formulations at 4 and 15 degrees C.

TABLE 20
Fenofibrate solubility in four formulations at 4 and 15 degrees C.

| | Formulation Composition (weight percent) | | | | Solubility (mg/mL) | |
|---|---|---|---|---|---|---|
| Formulation | E681010 | Ethanol | Cremophor EL | Span 20 | 4 deg C. | 15 deg C. |
| E | 65.0 | 15.0 | 10.0 | 10.0 | 70 | 115 |
| F | 70.0 | 10.0 | 10.0 | 10.0 | 72 | 107 |
| G | 64.9 | 10.0 | 12.5 | 12.6 | 70 | 95 |
| H | 65.0 | 8.5 | 13.3 | 13.3 | 68 | 94 |

### Example 14

### Physical Stability Characterization

Table 21 includes precipitation and resolubilization times for two fenofibrate formulations. For this study, both formulations were incubated at 4 degrees C. and observed for precipitation of fenofibrate. Both formulations J and K precipitated fenofibrate after 2 days. Following such precipitation, the formulations were brought to room temperature and the duration for resolubilization was observed. Formulation J took 2 days to resolubilize at room temperature while formulation K took at least 7 days to resolubilize. Formulations J and K were also incubated at 15 degrees C. and did not precipitate after 14 days.

TABLE 21
Physical stability study of fenofibrate in two formulations

| | Formulation Composition (weight percent) | | | | |
|---|---|---|---|---|---|
| | | | | Cremophor | |
| Formulation | E681010 | Ethanol | Fenofibrate | EL | Span 20 |
| J | 58.9 | 13.6 | 9.3 | 9.1 | 9.1 |
| K | 63.3 | 9.0 | 9.6 | 9.0 | 9.0 |

### Example 15

### Temperature Dependence of Fenofibrate Solubility in 85:15 E681010:Ethanol

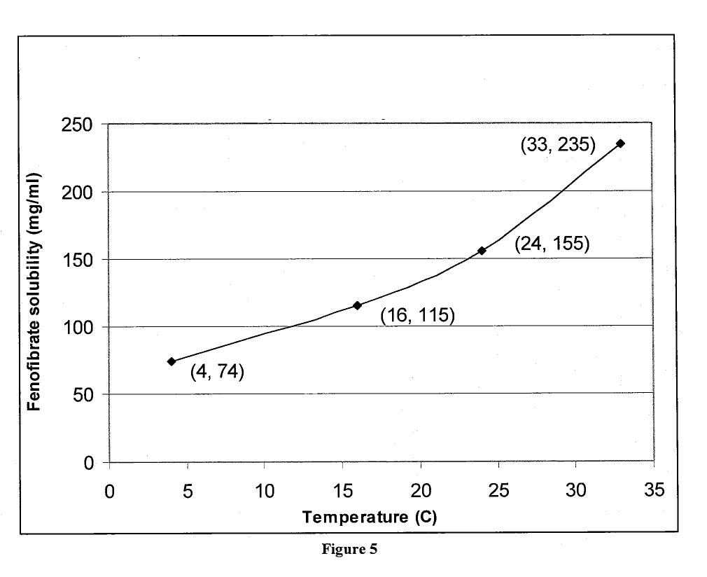

The solubility of fenofibrate was tested in 85:15 E681010:Ethanol (v/v) formulations at several temperatures. The fenofibrate dissolved in the vehicle and yielded a clear solution. The equilibrium solubility of fenofibrate at 25 degrees C. was approximately 160 mg/mL. FIG. 5 shows steep temperature dependence of fenofibrate solubility in the 85:15 E681010:Ethanol (v/v) vehicle.

What is claimed is:

1. A liquid formulation comprising fenofibrate dissolved in a vehicle comprising an omega-3 ester or omega-3 alkyl ester and a C₁ to C₄ alcohol, wherein: the formulation comprises (i) about 5% to about 20% by weight of fenofibrate; (ii) about 55% to about 85% by weight of an omega-3 ester or omega-3 alkyl ester; (iii) about 5% to about 20% by weight of a C₁ to C₄ alcohol; and (iv) about 5% to about 25% by weight of a surfactant.

2. The liquid formulation of claim 1, wherein the solubility of the fenofibrate in the vehicle is about 50 mg/mL to about 200 mg/mL.

3. The liquid formulation of claim 1, wherein the formulation comprises EPA and DHA in an amount which is between about 70 and about 90 percent by weight.

4. The liquid formulation of claim 1, wherein the omega-3 ester or omega-3 alkyl ester has a ratio of EPA:DHA from about 3:1 to about 1:1.

5. The liquid formulation of claim 1, wherein the omega-3 ester or omega-3 alkyl ester has a ratio of EPA:DHA from about 10:1 to about 5:1.

6. A method of increasing the solubility of fenofibrate in an omega-3 oil, comprising adding a C₁ to C₄ alcohol to said omega-3 oil.

7. The method of claim 6, wherein the solubility of fenofibrate is increased by at least about 50 percent.

8. The method of claim 6, wherein the omega-3 oil is an omega-3 ethyl ester.

9. The method of claim 6, wherein the alcohol is ethanol.

10. The method of claim 6, wherein the alcohol comprises from about 5 percent to about 20 percent by weight of the total formulation.

11. A liquid formulation comprising fenofibrate dissolved in a vehicle comprising an omega-3 ester or omega-3 alkyl ester and a C₁ to C₄ alcohol, wherein:
(a) the formulation comprises (i) about 5% to about 20% by weight of fenofibrate (ii) about 55% to about 85% by weight of an omega-3 ester or omega-3 alkyl ester, and (iii) about 5% to about 20% by weight of a C₁ to C₄ alcohol;
(b) the solubility of the fenofibrate in the vehicle is from about 50 mg/mL to about 200 mg/mL at 25 degrees C.; and (c) the liquid formulation does not contain a surfactant.

12. A method for treating hypercholesterolemia, atherosclerosis, hypertriglyceridemia, cardiovascular disease, coronary artery disease or cerebrovascular disease in a subject in need thereof, comprising administering to the subject an effective amount of the formulation of claim 1.

13. A method for treating hypercholesterolemia, atherosclerosis, hypertriglyceridemia, cardiovascular disease, coronary artery disease or cerebrovascular disease in a subject in need thereof, comprising administering to the subject an effective amount of the formulation of claim 11.

14. Fenofibrate Form II

15. The fenofibrate Form II of claim 14, wherein said Form II exhibits a powder X-ray diffractogram comprising peaks at about 12.51, 15.43, and 19.13 degrees 2-theta.

16. The fenofibrate Form II of claim 14, wherein said Form II exhibits a powder X-ray diffractogram substantially as shown in FIG. 1.

17. The liquid formulation of claim 14, wherein the formulation comprises EPA and DHA in an amount which is between about 70 and about 90 percent by weight.

18. The liquid formulation of claim 14, wherein the omega-3 ester or omega-3 alkyl ester has a ratio of EPA:DHA from about 3:1 to about 1:1.

19. The liquid formulation of claim 14, wherein the omega-3 ester or omega-3 alkyl ester has a ratio of EPA:DHA from about 10:1 to about 5:1.

20. A composition comprising the fenofibrate Form II of claim 14 and a carrier.
